(19) United States
(12) Patent Application Publication
(10) Pub. No.: US 2005/0196467 A1
(43) Pub. Date: Sep. 8, 2005

(54) Treatment system including carotenoid, nicotinamide, zinc, water soluble extract of uncaria species and deterium reduced aqueous fluid and method using the same

(76) Inventor: Vincent Giampapa, Montclair, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/046,108

(22) Filed: Jan. 28, 2005

Related U.S. Application Data
(60) Provisional application No. 60/540,898, filed on Jan. 30, 2004.

(51) Int. Cl. A61K 31/7076; A61K 31/455; A61K 31/405; A61K 35/78; A61K 31/015; A61K 33/32

(52) U.S. Cl. 424/641; 424/725; 514/355; 514/47; 514/419; 514/763

(57) ABSTRACT

A treatment system for administering to a human or other animals and the method of use for improving resistance to DNA damage, enhancing DNA repair capacity and stimulating immune function are provided. The treatment system includes a carotenoid material; a nicotinamide material, a zinc source material, a water soluble extract material of an Uncaria species and a deuterium-reduced aqueous fluid containing 0.1 to 110 ppm deuterium.

### CROSS REFERENCE TO RELATED APPLICATIONS

This application claims the benefit under 35 USC 119 (e) of the provisional patent application Ser. No. 60/540,898, filed on Jan. 30, 2004, which is herein incorporated by reference in its entirety.

### FIELD OF THE INVENTION

This invention relates to new and improved treatment system for and methods of treating humans and other animals to reduce DNA damage, enhance DNA repair capacity, and enhance immune function. More particularly, it relates to the administration to human (or other animal) subjects of a carotenoid material, a nicotinamide material, a zinc source material, a water soluble extract material of an Uncaria species and a deuterium reduced aqueous fluid material as a drug treatment or a daily dietary supplement, and to treatment system containing that combination of materials.

### BACKGROUND OF THE INVENTION

The exact mechanism of action of carotenoids such as beta carotene is not fully understood but it is commonly accepted scientifically that one primary mechanism is to scavenge oxygen derived free radicals produced either as by-products of metabolism or from exogenous environmental exposures. As a free radical scavenger, carotenoids can be expected to reduce or protect against the chemical damage induced in DNA, RNA and protein of cells by toxic environmental exposures or endogenous cellular metabolic errors that ultimately can result in a disease state.

Nicotinamide and its metabolic equivalent nicotinic acid (niacin, vitamin B₃) or even tryptophane which is the synthetic precursor to niacin are the main precursor for the formation and maintenance of the cellular pool of nicotinamide adenine dinucleotide (NAD). NAD is essential for cellular ATP production and maintenance of the cell's redox potential, and it is also the substrate for the DNA repair enzyme, poly ADP-ribosyl transferase (ADPRT). Niacin deprivation decreases the NAD pools significantly both in tissue culture cells, animals and humans. The depleted cells have an increased sensitivity to DNA damage, and the levels of poly(ADP-ribose) production in cultured cells or in rat liver were significantly lower after mild nicotinamide deficiency. On the other hand, when niacin was given as a supplement to ordinary nutrition (i.e. above known dietary levels) the NAD pool increased and the cells were less sensitive to oxygen radicals. Therefore, the primary mechanism of action of nicotinamide/niacin differs from carotenoids in that the cell's potential for energy metabolism is increased by amplifying NAD and ATP pool supplies (i.e. these biochemicals are the energy sources of living organisms) which in turn is useful to cells, tissues and organs to reduce DNA damage, enhance DNA repair (i.e. poly ADP-ribosylation) and stimulate immune function where the relevance to the disease state is apparent.

Zinc differs from the carotenoids and nicotinamide with regard to its mechanism of action in that it influences disease development and immune function by being an essential co-factor in several enzyme functions involving replication, DNA repair and antioxidant defense of cells. Zinc is required for cell replication and DNA polymerase activity. There are two zinc fingers in the DNA binding domain of the poly adenosine diphosphate ribosyl transferase (ADPRT) gene and other DNA repair proteins which contain cysteine residues, and if these cysteine residues are oxidized at their thiol constituents, they would prevent DNA binding and participation in DNA repair. Moreover, superoxide dismutase is an antioxidant enzyme protecting cells from the harmful superoxide anion. This radical is a substrate for the enzymatic reaction that also requires zinc as a cofactor.

Furthermore, U.S. Pat. No. 6,020,351 (to Pero) teaches the use of a combination of carotenoids, nicotinamide, and zinc, in the absence of other active components, to reduce DNA damage, enhance DNA repair capacity, and enhance immune function.

As taught in U.S. Pat. Nos. 6,039,949, 6,238,675 and 6,361,805 (to Pero), the water soluble extract of an Uncaria species known as C-MED-100® or Activar AC-11™ is known to give profound nutritional support as a dietary supplement because the water soluble extract of an Uncaria species enhances both DNA repair and immune cell responses, which, in turn, are the critical physiological processes that regulate aging. Both of these processes involve regulating the nuclear transcription kappa beta (NF-kB). NF-kB is well known to control (i) the nuclear events that salvage cells from apoptotic cell death and (ii) pro-inflammatory cytokine production. (Beg, A A and Baltimore, D., An essential role for NF-kB in preventing TNF-α induced cell death. Science 274: 782-784, 1996; Wang, C—Y, Mayo, M. W., Baldwin, A. S., TNF-α and cancer therapy-induced apoptosis: Potentiation by inhibition of NF-KB. Science 274: 784-787, 1996). Hence, this mechanism directly connects induction of apoptosis to programmed cell toxicity with inhibition of pro-inflammatory cytokine production and inflammation, which is different from the mechanisms of the above-described three chemicals.

On the other hand, as taught in U.S. Pat. Nos. 5,855,921 and 5,788,953 (to Somlyai) deuterium depleted water having deuterium concentration from 0.1 to 110 ppm inhibits tumor growth in animal model studies. It has also been reported in human clinical trials involving hundreds of cancer patients in Hungary that deuterium depleted water extended life span of cancer patients. It is believed that the deuterium is a component of a submolecular regulating system, and the processes temporarily elevating the concentration of deuterium trigger cell proliferation. Somlyai teaches that by applying water or aqueous solutions containing deuterium in an amount less than the deuterium content of natural water, the deuterium level in the human organism can be decreased as a result of exchange processes and in this way the proliferation of tumorigenic cells can be stopped or the development of cancerous tumors can be prevented. However, Somlyai does not teach or recognize whether deuterium depleted water is related to DNA repairing process.

It is known that in nature the ratio of hydrogen to deuterium is about 6000:1. Because of the mass difference of 100% the two isotopes show different behavior in chemical reactions. It is a generally accepted view that the D-bonds participating in chemical reactions can be split at a lower rate than the H-bonds because of the isotope effect, therefore they need an augmented activation energy. In enzymatic reactions it can be similarly measured that the reaction rate is of from 4 to 5 times higher with the hydrogen isotope having the smaller mass number.

A world-wide survey of hydrogen isotopes in precipitations revealed that the deuterium content is in a range of 120-160 ppm depending mainly on the site of sampling. According to the measurements, the deuterium content of the rainfall on the tropics is 155-160 ppm, whereas this value is only 120-150 ppm in the temperate zones of the world.

As shown above, each individual component has a different mechanism of action at cellular or molecular level for enhancing cell normal functions. However, in the prior art the above-described five materials have not been used, nor recognized, in a composition or as a system, to obtain enhanced functions in improving resistance to DNA damage, enhancing DNA repairing capacity and immune function, and preventing aging related disorders.

### SUMMARY OF THE INVENTION

In one aspect, the present invention is directed to a treatment system for administering to a mammal for improving resistance to DNA damage, enhancing DNA repair capacity and stimulating immune function. The treatment system consists essentially of a carotenoid material, a nicotinamide material, a zinc source material, a water soluble extract material of an Uncaria species and a deuterium-reduced aqueous fluid which has a deuterium concentration in a range from 0.1 to about 110 ppm.

In another aspect, the present invention is directed to a method of use of the treatment system of the present invention. The method consists essentially of administering to the subject, substantially daily, a carotenoid material, a nicotinamide material, a zinc source material, a water soluble extract material of an Uncaria species, and a deuterium-reduced aqueous fluid; said materials and fluid being administered to the subject in a daily dosage amounts effective, in combination, to improve resistance to DNA damage, enhance DNA repair capacity, and stimulate immune function.

### DETAILED DESCRIPTION OF THE INVENTION

In one embodiment, the present invention provides a treatment system which consists essentially of a carotenoid material, a nicotinamide material, a zinc source material, a water soluble extract material of an Uncaria species and an aqueous fluid having a deuterium concentration substantially below a naturally occurring deuterium level in water. The treatment system is preferably administered daily to increase an individual's resistance to cellular DNA damage, enhance cellular DNA repair and stimulate immune cell responsiveness in vivo.

The basic principle of this invention is to combine substances with known properties to improve resistance to DNA damage, enhance DNA repair capacity, stimulate immune function and prevent cancer, but with differing mechanisms of action. More specifically, carotenoids are electrophilic scavenger of radicals produced endogenously by cells or exogenously by the environment. Nicotinamide is amplified source of energy via increased production of NAD or ATP. Zinc is an essential cofactor to antioxidant, replicative and DNA repair enzymes in cells. The water soluble extract material of an Uncaria species prevents free radical damage by NF-kB inhibition, induces differentiation and immune cell responsiveness by apoptosis, enhances DNA repair, and kills tumor cells, which in turn are the major factors related to aging. The deuterium-reduced aqueous fluid reduces D₂O concentration in cellular environment. Since the deuterium bond in D2O is stronger than the hydrogen bond in H₂O, reducing concentration of D2O in cellular environment is expected to reduce the activation energy required in unwinding the DNA helix, hence it facilitates DNA repairing process. In combination, these five different mechanisms can synergistically achieve improvements of resistance to DNA damage, enhancement of DNA repair capacity and immune function, and prevention of aging related disorders.

The term “carotenoid material” as used herein means carotenoids, such as one or more materials selected from the group consisting of alpha carotene, beta carotene, gamma carotene, lycopene and mixtures thereof. The term “nicotinamide material” as used herein means one or more materials selected from the group consisting of nicotinamide, niacin, tryptophane (an amino acid precursor to niacin synthesis) and mixtures thereof. The term “zinc source material” as used herein means an appropriate source of zinc for administration to humans and/or other animals, e.g. one or more zinc salts, such as zinc sulfate or other zinc salts like amino acids such as methionine or aspartate, dipeptides, gluconates, halides, nitrates, oxides or acetates. The term “water soluble extract material of an Uncaria species” used herein means the water soluble extract material of an Uncaria species obtained using the method described in U.S. Pat. Nos. 6,361,805, 6,238,675 and 6,039,949 (to Pero), which are hereby incorporated by reference in their entireties. The water soluble extract material of an Uncaria species is also referred to as C-Med 100® in the literature. The term “deuterium-reduced aqueous fluid” as used herein means an aqueous fluid having a deuterium concentration substantially below a naturally occurring deuterium level in water, more specifically, having a deuterium level in a range from about 0.1 to about 110 ppm.

In illustrative or preferred embodiments of the present invention, the carotenoid material can be selected from the group consisting of alpha carotene, beta carotene, gamma carotene, lycopene and mixtures thereof; the nicotinamide material can be selected from the group consisting of nicotinamide, niacin, tryptophane and mixtures thereof; and the zinc source material can be one or more zinc salts. These materials can be obtained commercially. One available supplier is C. E. Jamieson, Ltd. (Ontario, Canada). The carotenoids are supplied as Caroplex in 100 mg soft gel capsules. Nicotinamide is in 100 mg tablets and zinc gluconate is in 10 mg tablets. Caroplex is a proprietary manufactured natural source of carotenoids from palm oil containing beta carotene=60%, alpha carotene=34%, gamma carotene=3% and Iycopene=3%. Other forms and dosages of these three materials are also available. The water soluble extract of an Uncaria species can be obtained commercially under the product name Activar AC-11™ from Optigenex, Inc, New York, N.Y.

The deuterium-reduced aqueous fluid can be used in various forms, such as deuterium-reduced water, fruit juice, soft drink, tea, coffee, sports drink and beer. The deuterium-reduced aqueous fluid can be made using deuterium-reduced water to produce fruit juice, soft drink, tea, coffee, sports drink and beer. The deuterium-reduced water can be produced by distillation or electrolysis, as described in U.S. Pat. Nos. 5,855,921 and 5,788,953, which are hereby incorporated by reference in their entireties. Using electrolysis, the deuterium concentration of the water can be reduced down to 30-40 ppm and further reduced to 6-20 ppm by a further electrolysis. Using distillation, the deuterium concentration of the water can be reduced down to 20-30 ppm and further reduced to 1-10 ppm by further increasing the plate number and/or repeating the distillation process. To produce a large volume of the aqueous fluid, this deuterium depleted water can be mixed with regular water in a predetermined proportion to obtain an aqueous fluid having a deuterium concentration from about 80 to about 110 ppm, which are substantially lower than the natural occurring level of deuterium in water and have been proved to be effective in reducing tumor size in animal model studies and in prolonging life span of cancer patients.

It should be understood that the treatment system of the present invention can be either formulated into one composition, such as a liquid form as described hereinafter, or the active components can be provided in two separate parts, with the deuterium-reduced aqueous fluid separated from four active components because of the large volume required for a substantially daily dosage as described hereinafter.

In one embodiment, the four active components other than the deuterium reduced aqueous fluid of the treatment system are combined into one composition. Alternatively, the four components can also be administered in the form of separate components. In either case, the four active components can be administered orally in solid or semi-solid dosage forms, such as for example hard or soft-gelatin capsules, tablets, and powders, or in liquid dosage forms, such as elixirs, syrups, dispersed powders or granules, emulsions, and aqueous or oily suspensions, together with the deuterium-reduced aqueous fluid, in the amounts described hereinafter.

Compositions intended for oral use can be prepared according to any methods known to the art for the manufacture of pharmaceutical compositions and such compositions can contain one or more agents including sweetening agents, flavoring agents, coloring agents, and preserving agents in order to provide a pharmaceutically elegant and palatable preparation. Tablets contain the active components in admixture with non-toxic pharmaceutically acceptable excipients which are suitable for the manufacture of tablets. Such excipients can include, for example, inert diluents, such as calcium phosphate, calcium carbonate, sodium carbonate, sodium phosphate, or lactose; granulating disintegrating agents, for example, maize starch or alginic acid; binding agents, such as starch, gelatin, or acacia; and lubricating agents, for example, magnesium stearate, stearic acids or talc. Compressed tablets can be uncoated or can be sugar coated or film coated by known techniques to mask any unpleasant taste and protect the tablet from the atmosphere, or enteric coated for selective disintegration and adsorption in the gastrointestinal tract. Hard gelatin capsules or liquid filled soft gelatin capsules contain the active components and inert powdered or liquid carriers, such as, but are not limited to calcium carbonate, calcium phosphate, kaolin, lactose, lecithin starch, cellulose derivatives, magnesium stearate, stearic acid, arachis oil, liquid paraffin, olive oil, pharmaceutically-accepted synthetic oils and other diluents suitable for the manufacture of capsules. Both tablets and capsules can be manufactured as sustained release-products to provide for continuous release of medication over a period of hours.

In a further embodiment, the four active components are mixed with a deuterium reduced water and suitable excipients to form a liquid composition such as an aqueous suspension. Excipients suitable for manufacturing aqueous suspensions include suspending agents, e.g., sodium carboxymethylcellulose, methylcellulose, hydroxypropylmethylcellulose, sodium alginate, polyvinylpyrrolidone, gum tragacanth, and gum acacia; dispersing or wetting agents, such as a naturally occurring phosphatide, e.g., lecithin, or condensation products of an alkylene oxide with fatty acids, for example of polyoxyethylene stearate, or a condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monooleate, or a condensation product of ethylene oxide with partial esters derived from fatty acids and hexitol anhydrides, e.g., polyoxyethylene sorbitan monooleate. The aqueous suspensions can also contain one or more preservatives, for example ethyl, n-propyl, or p-hydroxy benzoate, one or more coloring agents, one or more flavoring agents, and one or more sweetening agents, such as sucrose, saccharin, or sodium or calcium cyclamate. Suitable pharmaceutical carriers are described in Remington's Pharmaceutical Sciences, Mack Publishing Company, a standard reference text in this field, which is incorporated herein by reference in its entirety.

Furthermore, the treatment system can also be produced in the form of a sterile injectable preparation, for example, as a sterile injectable aqueous suspension. This suspension can be formulated according to the known art using those suitable dispersing or wetting agents and suspending agents which have been mentioned above.

In a further aspect, the present invention provides a method of using the treatment system to reduce DNA damage, improve DNA repairing processes, enhance immune function, and prevent aging related disorders.

In a preferred embodiment, the treatment system is orally administered substantially daily. The daily dosage of the treatment system is from about 50 to 150 mg of the carotenoid material, from about 50 to about 150 mg of the nicotinamide material, from about 5 to about 15 mg of a zinc salt, from about 250 to about 1000 mg of the water soluble extract of an Uncaria species in the form of AC-11™, and 500 ml or more, preferably, 1 liter or more of deuterium reduced aqueous fluid which contains deuterium in a concentration range of from 0.1 ppm to 110 ppm.

It is noted that the dosage volume of the deuterium reduced aqueous fluid is reversely proportional to the concentration of the deuterium. In general, the objective is to reduce the deuterium level inside the body of a subject who takes the treatment system of the present invention, by 0.5 ppm or more.

As described above, the treatment system can be in one composition or in two parts. Due to its large volume for oral administration, the deuterium reduced aqueous fluid is administered orally multiple times in a day. Furthermore, the deuterium reduced aqueous fluid can be in various different forms as described above.

The effects of the treatment method of the present invention on a human and other mammalian subject can be determined by monitoring one or more of the following responses to the treatment by blood analyses: an increase of human mononuclear leukocyte's ability to resist H₂O₂ induced DNA damage, an increase of DNA repairing of H₂O₂ induced DNA damage at certain time intervals, enhancement of poly ADPRT activity, an increase of NAD cellular concentration of erythrocytes, and enhancement of PHA-induced mitogenic response. One or more these improvements indicate improved resistance to DNA damage, enhanced DNA repair capacity and immune function, which, in turn, leads to the reduction of aging related diseases.

The blood analyses for determining human mononuclear leukocyte's ability to resist H₂O₂ induced DNA damage, DNA repairing of H₂O₂ induced DNA damage, poly ADPRT activity, NAD cellular concentration of erythrocytes, and PHA-induced mitogenic response are described in details in U.S. Pat. No. 6,020,351, which, and the various references cited therein, are hereby incorporated by reference in their entireties.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A treatment system for administering to a mammal consisting essentially of:
(a) a carotenoid material;
(b) a nicotinamide material;
(c) a zinc source material;
(d) a water soluble extract material of an Uncaria species; and
(e) a deuterium-reduced aqueous fluid.

2. The treatment system of claim 1, wherein said deuterium-reduced aqueous fluid has a deuterium concentration in a range from 0.1 to about 110 ppm.

3. The treatment system of claim 2, wherein said nicotinamide material is at least one selected from the group consisting of nicotinamide, niacin, tryptophane, NAD, NADH, NADP, NADPH and combination thereof

4. The treatment system of claim 2, wherein said carotenoid material is at least one selected from the group consisting of alpha carotene, beta carotene, gamma carotene, lycopene and combination thereof.

5. The treatment system of claim 2, wherein said zinc source material is one or more zinc salts.

6. The treatment system of claim 1 further consisting of one or more pharmaceutically acceptable excipients.

7. A method of treating a human or other animal subject consisting essentially of administering to the subject, substantially daily, a carotenoid material, a nicotinamide material, a zinc source material, a water soluble extract material of an Uncaria species, and a deuterium-reduced aqueous fluid; said materials and fluid being administered to the subject in a daily dosage amounts effective, in combination, to improve resistance to DNA damage, enhance DNA repair capacity, and stimulate immune function.

8. The method of claim 7, wherein said daily dosage includes about 50 to 150 mg of said carotenoid material, about 50 to about 150 mg of said nicotinamide material, about 5 to about 15 mg of said zinc source material, about 250 to about 1000 mg of said water soluble extract of Uncaria species, and 500 ml or more of said deuterium-reduced aqueous fluid.

9. The method of claim 7, wherein said deuterium-reduced aqueous fluid has a deuterium concentration in a range from 0.1 to about 110 ppm.
